(19) United States
(12) Patent Application Publication
(10) Pub. No.: US 2026/0263332 A1
(43) Pub. Date: Sep. 10, 2026

(54) SUN PROTECTION DEVICES AND METHODS

(71) Applicants: Zachary BOLDUC, Salisbury, MA (US); Roy Alan BOLDUC, Salisbury, MA (US); Douglas HEMPLEPP, Salisbury, MA (US)

(72) Inventors: Zachary BOLDUC, Salisbury, MA (US); Roy Alan BOLDUC, Salisbury, MA (US); Douglas HEMPLEPP, Salisbury, MA (US)

(21) Appl. No.: 19/165,633

(22) PCT Filed: Mar. 15, 2024
(86) PCT No.: PCT/US2024/020259

§ 371 Date: Sep. 16, 2025

Related U.S. Application Data
(60) Provisional application No. 63/490,574, filed on Mar. 16, 2023.

(51) Int. Cl. A61K 8/11; A61K 8/66; A61Q 17/04

(52) U.S. Cl. CPC A61K 8/11; A61K 8/66; A61Q 17/04; A61K 2800/26; A61K 2800/262

(57) ABSTRACT

A product for use in treating a subject, incorporating a light-conversion medium which receives ambient light of broad spectrum, and emits light at one or more peak wavelengths or ranges of wavelengths (λ1, λ2, λ3) having greater intensity than corresponding intensity in the received broad spectrum ambient light. The products outlined are specifically designed for UV protection which can be targeted to treat skin treatments of many different kinds, where the wavelengths chosen to have therapeutic or enhancing effects to the biological processes that occur inside or that directly affect the skin. The products can either exist as a unique treatment or be coupled with existing treatments in order to achieve an improved or differentiating result.

### FIELD OF THE INVENTION

The present invention is directed to apparatus, systems and methods for providing protection from ultraviolet light treatments to a subject in need thereof.

### BACKGROUND OF THE INVENTION

Sun protection, alternatively referred to as UV protection, involves the use chemicals and physical particles to absorb or reflect ultraviolet radiation. For example, existing sun protection formulations sometimes use physical UV absorbers like Zinc Oxide in a skin cream to provide UV protection in humans.

At present, commercially available UV protection technologies typically incorporate one or more established UV absorbers, such as Zinc Oxide, to absorb or reflect incoming UV and protect the subject from UV radiation by some factor. For example, many existing commercial products utilize, either individually or in combination, different organic molecules that contain chemical bonds that absorb UV radiation. While effective, such treatments have come under intense scrutiny due to their potential health and environmental hazards. When considering UV protection, different bands have been established in industry where the two primary bands are known as UVA and UVB. The range of UVB is 280 nm to 315 nm and UVA ranges from 315 nm to 400 nm. Existing commercial UV protection products may protect against either UVB alone or both UVB and UVA.

The inventors have developed compositions, platforms, systems and methods directed to improved UV protection of mammals, including humans.

### SUMMARY OF THE INVENTION

In one or more implementations, the present invention is directed to one or more light conversion devices that receive and absorb the UV spectrum, whether sunlight or artificial sources, and emit light at one or more peak wavelengths, or ranges of wavelengths. In a particular implementation, the emitted wavelengths or wavelength ranges have greater intensity than the corresponding intensity of the wavelength in the received broad spectrum ambient light. However, importantly, these light conversion devices convert UVA and UVB light into wavelengths that pose less of a concern to the health of an individual.

In one or more further particular implementations, one or more phototherapy platforms are configured to deliver phototherapy to a subject. The phototherapy platform is configured with at least one emission compound configured to receive light across a broad spectrum, including the UV spectrum, and emit at least a portion of the total wavelength of the received broad spectrum, wherein the emitted portion of the wavelength is emitted at a greater intensity than the intensity of light within the corresponding portion of the broad spectrum received.

In yet a further implementation, an emissive compound is configured to emit the absorbed light across a first wavelength range and emit light at a second wavelength range that is not within the first wavelength range.

In yet a further implementation, an emissive compound contains one or more quantum dots tuned to emit a specific emission spectrum that mirrors at least the absorption spectra for Cytochrome C Oxidase or other transmembrane protein complexes that are involved in cellular respiration, repair or regulation.

In a particular implementation, the foregoing disclosure relates to products for phototherapy or photoprotection, including compositions incorporated into topical creams, gels, and ointments. Such products for UV protection and photoprotection can be used in one or more methods of treating skin treatments, including but not limited, UV damage, DNA damage, wrinkle elimination or reduction.

### BRIEF DESCRIPTION OF THE DRAWINGS

The subject matter disclosed herein is, in part, illustrated in the figures of the accompanying drawings which are meant to be exemplary and not limiting, in which like references are intended to refer to like or corresponding parts, and in which:

FIG. 1 represents sun protection phototherapy treatment in accordance with the subject matter described herein.

FIG. 2 represents sun protection phototherapy treatment in accordance with the subject matter described herein.

FIG. 3 provides a chart detailing the performance of the described compositions relative to other compositions.

### DETAILED DESCRIPTION OF CERTAIN EMBODIMENTS OF THE INVENTION

By way of overview and introduction, various embodiments of the compositions and methods described herein are directed towards UV protection and photoprotection platform. In a particular implementation of the UV protection and photoprotection platform described, the UV protection and photoprotection platform is implemented as one or more compositions designed to produce a given wavelength range of light when exposed to UV radiation contained within ambient light. In yet a further particular implementation, the UV protection and photoprotection platform includes one or more films, filters, gels, creams and ointments containing emissive compounds that can be applied to the skin or other biological surface of a subject, such as a human, as part of a complete skin treatment or as an any UV protection measure.

In a further particular implementation, the UV protection and photoprotection platforms include one or more encapsulated quantum dot formulations configured to emit light at a wavelength range suitable for use in phototherapy.

In a further particular implementation, a method is provided for delivering a combination of photoprotection and phototherapy to a subject using one or more encapsulated quantum dots. In a particular implementation, the UV protection and photoprotection platform and methods of using the same provided herein are directed to providing therapy or otherwise providing phototherapy for skin treatments. For example, the wavelengths emitted by the UV protection and photoprotection platform are useful for treatment of UV damage, wrinkles, and scars present on the skin of an individual.

In one or more implementations, the foregoing provides one or more methods of providing a UV protection and photoprotection platform to a person in need of treatment wherein the method includes the application of a fluid, gel or cream UV protection and photoprotection platform as a treatment for UV protection and the skin ailments described herein.

In one or more particular implementations of the compositions and methods provided, a composition is provided whereby the composition includes a plurality of quantum dots, each quantum dot encapsulated in an encapsulation compound transparent to a first wavelength spectrum, wherein the quantum dot is configured to absorb ambient light within the first wavelength range and emit light within a second wavelength range, where the second wavelength range is encompassed by the first wavelength range, wherein a first portion of the plurality of quantum dots are configured to emit light within the second wavelength range that is different from the light emitted by a second portion of the plurality of quantum dots.

In another implementation or embodiment, a composition is provided wherein the composition is directed for use in protecting from UV radiation damage, deficiency or non-aesthetic symptom, incorporating a light-conversion medium which receives ambient light of broad spectrum which contains UV radiation, and emits light at one or more peak wavelengths or ranges of wavelengths (λ1, λ2, λ3) having greater intensity than corresponding intensity in the received broad spectrum ambient light.

The quantum dot composition as provided herein is configured to emit light at one or more peak wavelengths or ranges of wavelengths λ1, λ2, λ3, wherein the peak wavelength ranges are from about 200 nm to about 3000 nm, optionally in the UV, optionally from about 200 nm to about 400 nm, optionally in the UVA, optionally from about 310 nm to about 360 nm, optionally in the visible, optionally from about 400 nm to about 600 nm, optionally in the IR, optionally from about 600 nm to about 3000 nm, optionally in the NIR, optionally from about 810 nm to about 850 nm.

By way of one particular implementation, the encapsulated quantum dots are configured to receive light in the 315-400 nm and/or 280-315 nm range and convert or emit light at 600-700 nm. In a further arrangement, the quantum dots are configured to absorb light from 250nm-500 nm. In one or more further arrangements, some quantum dots are configured to emit light between 500-700 nm range. In a further arrangement, light is emitted by the one or more of the quantum dots included in the composition is between 630-660 nm.

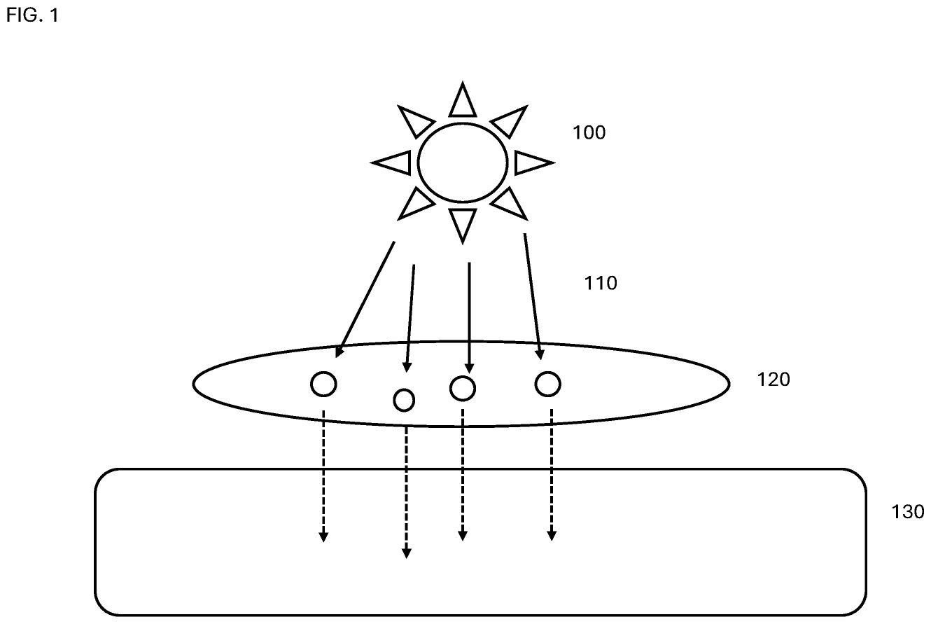

As shown with respect to FIG. 1, a light source 100, such as natural light or artificial light emits light at one or more wavelengths 110. Those wavelengths are absorbed by the quantum dots of the described composition 120. The quantum dots then emit light at one or more different wavelengths compared to the original incoming wavelength (shown in dashed lines) to the skin 130 of a user.

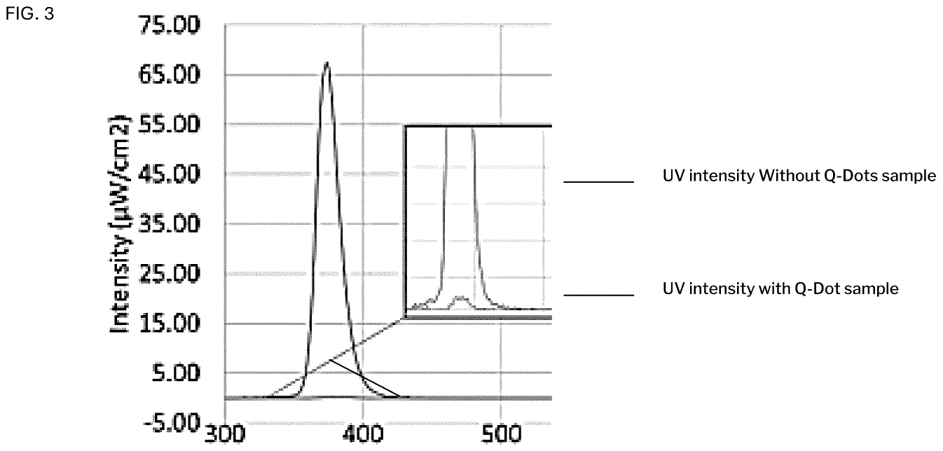

As shown in the graph of FIG. 3, in one or more configurations the described silicon based quantum dots are configured to absorb UV light compared to a sample that lacks Q-Dots.

In one or more further arrangements, the composition include glycerol. For example, in one or more configurations, the composition includes silicon-based quantum dots encapsulated in glycerol.

In one or more further configurations, the described formulations of encapsulated quantum dots may also include a UV absorber or blocker. In a further arrangement, the described composition may include one or more pigments and, or emollients.

By way of further, non-limiting example, the compositions described herein include suspensions or emulsions or titanium dioxide with organic sunscreen compounds. For example, and in no way limiting, the compositions described can include one or more of the following: aminobenzenic acid, avobenzone, cinoxate, dioxybenzone, homosalate, menthyl anthronilate, octocrylene, octyl methoxycinnamate, octyl salicylate, oxybenzone, padimate, phenylbenzinadazole sulfonic acid, sulisbenzone, trolamine salicylate and zinc oxide.

By way of further, non-limiting example, the compositions described herein include one or more adjuvants for topical or gel base forms which include one or more of sodium carboxymethylcellulose, polyacrylates, polyoxyethylene-polyoxypropylene-block polymers, polyethylene glycol, wood wax alcohols, isostearic acid, cetyl alcohol, stearyl alcohol, white petrolatum, polysorbate 60, sorbitan monostearate, glycerin, xanthan gum, water, benzyl alcohol, methylparaben, and propylparaben.

By way of further, non-limiting example, the compositions described herein include one or more additional additives selected from the group consisting of waxes, soaps, sorbitan esters, fatty acids, fatty acid esters, fatty acid oils, borates, cresol, chlorocresol, cellulose, methylcellulose, hydroxypropylcellulose, acacia, and the like.

In yet further arrangements, the compositions described herein include provide a sun protection factor of SPF 15, 30, 50 or more and is in the form of a liquid (e.g., liquid lotion, liquid serum), cream or gel (e.g., ointment gel). In further arrangements, the SPF factor provided by the described formulations is no less than 60 SPF.

In yet another arrangement, the effective amounts of the encapsulated quantum dots within a composition are in a range of 0.003-5%. In a further arrangement, the formulation includes silicon quantum dots in the range of 0.003-5%, zinc oxide in a range of 5-15% and titanium dioxide in a range of 10-15%. The ranges may be provided as % m/m, % w/w, % w/v % m/v, or % v/v of the formulation.

In yet a further arrangement, the composition contains one or more collagenases, or other collagen dissolving enzymes. For example, the composition may contain one or more of collagenase 1 (MMP-1), collagenase 2 (MMP-8), collagenase 3 (MMP-13), and collagenase 4 in Xenopus (MMP-18).

For example, a composition is provided of any of the described compositions wherein the formulation further includes encapsulated silicon quantum dots in the range of 0.0003-5%. In a further arrangement, the composition is 5% encapsulated quantum dot material. In a particular configuration, the formulation further includes encapsulated silicon quantum dots in the range of 0.003-0.5%. In a particular configuration, the formulation further includes encapsulated silicon quantum dots in the range of 0.03-0.5%. In a particular configuration, the formulation further includes encapsulated silicon quantum dots in the range of 0.1-0.5%.

In a further composition, zinc oxide in a range of 5-15% is provided. In yet a further composition, titanium dioxide in a range of 10-15% is provided. In yet a further composition, at least 0.001-5% collengase is provided.

The ranges may be provided as % m/m, % w/w, % w/v % m/v, or % v/v of the formulation. In yet a further arrangement, the pH of the composition is between 6 and 8.

In one or more further implementations the quantum dots are configured to have inhibited or muted fluorescence. In this configuration, the quantum dots are configured to have UV absorption properties, but not to emit light within the described wavelengths. In one configuration, such fluoresce fouling is implemented by encapsulating the quantum dot in material that is configured to absorb the wavelengths of light generated by the quantum dot, but is transparent to the wavelengths of light that the quantum dot is configured to absorb.

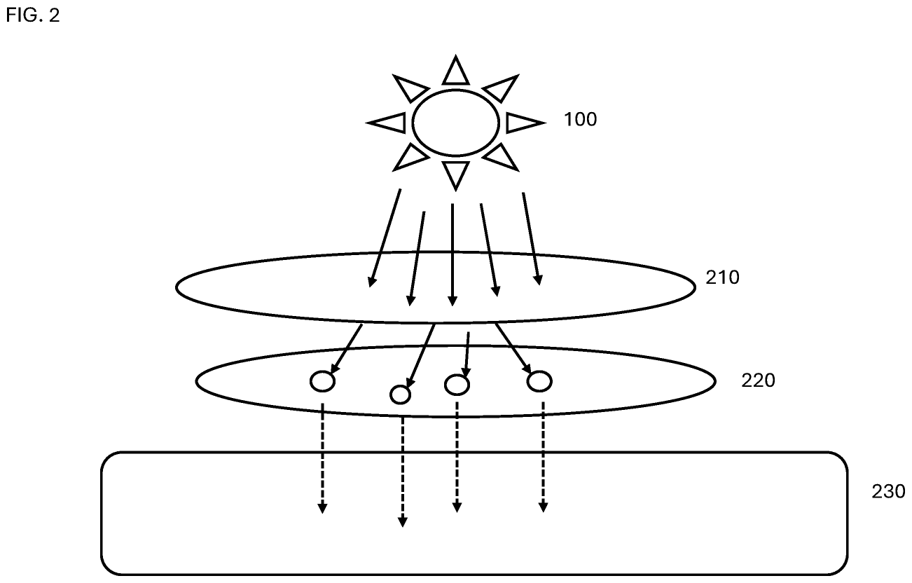

In a further particular arrangement, a composition of any of the previously provided compositions is provided whereby the light-conversion medium comprises a carrier medium and a light conversion element. In one or more implementations, the composition is combined with one or more UV filters. For example, as shown in FIG. 2, quantum dot formulation 220 described herein is provided to a substrate, such as glass or plastic. However, an additional filter material, such as UV filters 210, can be combined with the substrate to provide a composite product. In this configuration, light from the light source 100 first passes through the filter material 210. This material absorbs some portion of the light at a given wavelength. This light then passes through the composition described herein, further absorbing light. The quantum dots emit light at a different wavelength (shown in dashed lines) to the user or wearer 230.

In a further particular arrangement, a composition of any of the previously provided compositions is provided whereby the particulate light-conversion medium is supported by a light-transmissive carrier or support medium, optionally the support medium is transparent, translucent, or opaque.

In a further particular arrangement, a composition of any of the previously provided compositions is provided whereby the particulate light-conversion medium is suspended in the support medium or molecularly bonded to the support medium, thus creating a new hybrid material.

In a further particular arrangement, a composition of any of the previously provided compositions is provided whereby the support medium is a solid material, such as a plastics material, a polymeric material, or an organic material, optionally the solid support medium is rigid, semi-rigid or flexible.

In a further particular arrangement, a composition of any of the previously provided compositions is provided whereby the support medium is a fluid material, such as a gel, a cream, or a liquid.

In a further particular arrangement, a composition of any of the previously provided compositions is provided whereby the fluid material includes one or more compounds includes one or more collagen dissolving enzymes.

In a further particular arrangement, a composition of any of the previously provided compositions is provided whereby the fluid material can be used as an additive to existing UV protection serums, creams or gels.

In a further particular arrangement, a composition of any of the previously provided compositions is provided whereby the product is a composition which can be applied directly to the subject, such as for application to the skin, for example, as a topical cream, gel, or ointment, (i) a bio-compatible material or intrinsically modified to be bio-compatible or (ii) encapsulated so as to prevent direct contact with the subject, optionally the encapsulated fluid support medium has the form of a gel, cream, or ointment for topical application to the skin of a human.

In a further particular arrangement, a composition of any of the previously provided compositions is provided whereby the skin ailment, deficiency, or non-aesthetic symptom addressed by the composition is scar tissue, existing UV damage, wrinkles, discoloration, erythema, solar erythema, and inflammation.

In a further particular arrangement, a composition of any of the previously provided compositions is provided whereby the product is a gel, cream, or ointment which is used, for example, in the simultaneous protection of UV and the treatment of a skin condition.

In a further particular arrangement, a composition of any of the previously provided compositions is provided whereby the composition can be coupled or mixed in with current treatment components, ingredients or methods in order to enhance or differentiate the existing product.

In a further particular arrangement, a composition of any of the previously provided compositions is provided whereby the light-conversion medium has a major fraction of particles having an average diameter of from about 0.1 nm to about 1000 nm, optionally from about 0.1 nm to about 100 nm, optionally from about 0.1 nm to about 10 nm, optionally from about 1 nm to about 10 nm, optionally from about 10 nm to about 100 nm.

In a further particular arrangement, a composition of any of the previously provided compositions is provided whereby the light-conversion medium comprises a nanocrystal or nanoparticle semiconductor.

In a further particular arrangement, a composition of any of the previously provided compositions is provided whereby the light-conversion medium comprises quantum dots or Q-dots (QDs).

In a further particular arrangement, a composition of any of the previously provided compositions is provided whereby the light-conversion medium includes single shell QDs, multi-shell QDs, heavy metal QDs and/or non-heavy metal QDs.

In a further particular arrangement, a composition of any of the previously provided compositions is provided whereby the light-conversion medium includes silicon quantum dots which are molecularly bonded to the transparent medium.

In a further particular arrangement, a composition of any of the previously provided compositions is provided whereby the light-conversion medium includes silicon quantum dots which are molecularly bonded to the transparent medium, wherein the ratio of silicon quantum dots to non-silicon quantum dots is 1:1.

In a further particular arrangement, a composition of any of the previously provided compositions is provided whereby the light-conversion medium includes silicon quantum dots which are molecularly bonded to the transparent medium, wherein the ratio of silicon quantum dots to non-silicon quantum dots is less than 1:1.

In a further particular arrangement, a composition of any of the previously provided compositions is provided whereby the light-conversion medium includes silicon quantum dots which are molecularly bonded to the transparent medium, wherein the ratio of silicon quantum dots to non-silicon quantum dots is greater than 1:1.

Further implementations and embodiments of the subject matter described herein can be combined with the disclosure of PCT/US21/18873 or U.S. Patent Publication No. 20230073172A1 both of which are herein incorporated by reference in their respective entireties.

While this specification contains many specific embodiment details, these should not be construed as limitations on the scope of any embodiment or of what can be claimed, but rather as descriptions of features that can be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this specification have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

Publications and references to known registered marks representing various systems cited throughout this application are incorporated by reference herein. Citation of any above publications or documents is not intended as an admission that any of the foregoing is pertinent prior art, nor does it constitute any admission as to the contents or date of these publications or documents. All references cited herein are incorporated by reference to the same extent as if each individual publication and references were specifically and individually indicated to be incorporated by reference.

While the invention has been particularly shown and described with reference to a preferred embodiment thereof, it will be understood by those skilled in the art that various changes in form and details may be made therein without departing from the spirit and scope of the invention. As such, the invention is not defined by the discussion that appears above, but rather is defined by the claims that follow, the respective features recited in those claims, and by equivalents of such features.

What is claimed is

1. A composition comprising:
a. a plurality of quantum dots, each quantum dot encapsulated in an encapsulation compound transparent to a first wavelength spectrum, wherein the quantum dot is configured to absorb ambient light within the first wavelength range and emit light within a second wavelength range, where the second wavelength range is encompassed by the first wavelength range, wherein a first portion of the plurality of quantum dots are configured to emit light within the second wavelength range that is different from the light emitted by a second portion of the plurality of quantum dots.

2. A composition for use in protecting from UV radiation damage, deficiency or non-aesthetic symptom, incorporating a light-conversion medium which receives ambient light of broad spectrum which contains UV radiation, and emits light at one or more peak wavelengths or ranges of wavelengths (λ1, λ2, λ3) having greater intensity than corresponding intensity in the received broad spectrum ambient light.

3. The composition of claim 2, wherein the one or more peak wavelengths or ranges of wavelengths λ1, λ2, λ3 are from about 200 nm to about 3000 nm, optionally in the UV, optionally from about 200 nm to about 400 nm, optionally in the UVA, optionally from about 310 nm to about 360 nm, optionally in the visible, optionally from about 400 nm to about 600 nm, optionally in the IR, optionally from about 600 nm to about 3000 nm, optionally in the NIR, optionally from about 810 nm to about 850 nm.

4. The composition of claim 2, wherein the light-conversion medium comprises a carrier medium and a light conversion element.

5. The composition of claim 4, wherein the particulate light-conversion medium is supported by a light-transmissive carrier or support medium, optionally the support medium is transparent, translucent, or opaque.

6. The composition of claim 5, wherein the particulate light-conversion medium is suspended in the support medium or molecularly bonded to the support medium, thus creating a new hybrid material.

7. The composition of claim 5, wherein the support medium is a solid material, such as a plastics material, a polymeric material, or an organic material, optionally the solid support medium is rigid, semi-rigid or flexible.

8. The composition of claim 5, wherein the support medium is a fluid material, such as a gel, a cream, or a liquid.

9. The composition of claim 8, wherein the fluid material includes one or more compounds includes one or more collagen dissolving enzymes.

10. The composition of claim 9, wherein the formulation includes silicon quantum dots in the range of 0.005-5%, zinc oxide in a range of 5-15% and titanium dioxide in a range of 10-15%. The ranges may be provided as % m/m, % w/w, % w/v, % m/v, or % v/v of the formulation.

11. The composition of claim 8, wherein the product is a composition which can be applied directly to the subject, such as for application to the skin, for example, as a topical cream, gel, or ointment, (i) a bio-compatible material or intrinsically modified to be bio-compatible or (ii) encapsulated so as to prevent direct contact with the subject, optionally the encapsulated fluid support medium has the form of a gel, cream, or ointment for topical application to the skin of a human.

12. A method of treating a skin ailment, deficiency, or non-aesthetic symptom, the method comprising administering a composition that includes at least a plurality of quantum dots, each quantum dot encapsulated in an encapsulation compound transparent to a first wavelength spectrum, wherein the quantum dot is configured to absorb ambient light within the first wavelength range and emit light within a second wavelength range, where the second wavelength range is encompassed by the first wavelength range, wherein a first portion of the plurality of quantum dots are configured to emit light within the second wavelength range that is different from the light emitted by a second portion of the plurality of quantum dots.

13. The method of claim 12, wherein the skin ailment, deficiency, or non-aesthetic symptom is one of scar tissue, existing UV damage, wrinkles, discoloration, erythema, solar erythema, and inflammation.

14. The product of claim 12, wherein the composition administered is in the form of a gel, cream, or ointment.

15. The product of claim 2, wherein the product can be coupled or mixed in with current treatment components, ingredients or methods in order to enhance or differentiate the existing product.

16. The product of claim 2, wherein the light-conversion medium has a major fraction of particles having an average diameter of from about 0.1 nm to about 1000 nm, optionally from about 0.1 nm to about 100 nm, optionally from about 0.1 nm to about 10 nm, optionally from about 1 nm to about 10 nm, optionally from about 10 nm to about 100 nm.

17. The product of claim 2, wherein the light-conversion medium comprises a nanocrystal or nanoparticle semiconductor.

18. The product of claim 15, wherein the light-conversion medium comprises quantum dots or Q-dots (QDs).

19. The product of claim 16, wherein the light-conversion medium includes single shell QDs, multi-shell QDs, heavy metal QDs and/or non-heavy metal QDs.

20. The product of claim 17, wherein the light-conversion medium includes silicon quantum dots which are molecularly bonded to the transparent medium.
